(19) United States
(12) Patent Application Publication
(10) Pub. No.: US 2003/0195588 A1
(43) Pub. Date: Oct. 16, 2003

(54) External ear canal interface for the treatment of neurological disorders

(75) Inventors: David R. Fischell, Fair Haven, NJ (US); Adrian R.M. Upton, Dundas (CA)

Correspondence Address: NEUROPACE, INC., 1375 SHOREBIRD WAY, MOUNTAIN VIEW, CA, 94043, US

(73) Assignee: NeuroPace, Inc., Sunnyvale, CA

(21) Appl. No.: 10/123,857

(22) Filed: Apr. 16, 2002

(51) Int. Cl. A61N 1/18

(52) U.S. Cl. 607/55

(57) ABSTRACT

A system for treating various neurological, vestibular, and other disorders includes a stimulator device situated in an ear canal of the patient. The stimulator device is adapted to provide magnetic, electrical, audible, tactile, or caloric stimulation, and may be programmed to provide such stimulation in continuous, semi-continuous, periodic, programmed, or on-demand modes, or various combinations of the above.

### FIELD OF THE INVENTION

This invention is in the field of external medical devices for the treatment of neurological disorders such as epilepsy, febrile seizures, dizziness, tinnitus and sleep disturbances.

### BACKGROUND OF THE INVENTION

Today most neurological disorders are treated with drugs that can have significant side effects. There are few existing devices for the treatment of neurological disorders that are not fully implanted. There are existing devices that use eye movement to indicate the onset of drowsiness and sound an auditory alerting signal. Current external devices do not include EEG, auditory, vestibular or caloric recording capability nor do they have electrical or caloric stimulation capability. Furthermore most external EEG or ECG monitoring devices are not capable of responsive interventions. Fischell and Upton in U.S. Pat. No. 6,016,449 (which is hereby incorporated by reference as though set forth in full herein) describe numerous embodiments of a responsive neurostimulator that can use responsive electrical and sensory (acoustic, vibratory and visual) stimulation applied externally to treat neurological disorders. Fischell and Upton, also describe an embodiment of their invention that could be completely external using “a control module located external to the patient's body connected to electrodes (either) external (or internal) to the patient's scalp. Such an externally located control module might be positioned behind the patient's ear like a hearing aid.” External scalp electrodes, however are not well suited for accurate detection of the onset of neurological states due to significant noise from artifacts such as muscle contraction, eye movement or sweating. As a result, most of the embodiments of the U.S. Pat. No. 6,016,449 utilize brain electrodes implanted under the scalp. Vestibular and caloric stimulation therapies were not mentioned at all as a potential responsive treatment for a neurological disorder.

The two major problems associated with sleep are inability to get to sleep and difficulty in staying awake. Sleep apnea is currently treated with surgery to the throat or airways, air flow devices that often do not work and are uncomfortable and poorly tolerated by many patients and by ear clip oxygen sensors with acoustic feedback that will wake the patient contributing to interrupted sleep. In the second category, disorders relating to difficulty in staying awake such as narcolepsy can cause inadvertent drowsiness or sleep that is extremely dangerous when driving or operating heavy equipment. Currently stimulants are used to help keep such people awake during the day but are often either ineffective or cause additional problems of affecting normal night time sleep. In addition, there are no current non-drug treatments for sleep disorders associated with changes in time zone associated with travel nor are there any device solutions for control of sleep stages.

There are currently available devices for detection of sleep stages used in sleep laboratories as part of night time EEG recordings. These devices are bulky, impractical for home use or direct therapeutic intervention.

Dizziness is a common and debilitating disorder that is largely treated with medication of destructive surgery to the inner ear. It is a major cause of disability for which no reliable current treatment exists.

Current devices for epilepsy are all fully implantable and do not include any form of vestibular stimulation. However the implantable Cyberonics device using vagal stimulation probably has effects on vestibular mechanisms.

Febrile seizures that occur particularly but not exclusively in children of 5 and below, are usually treated with cooling of the child's body or anti-fever drugs after the fever is established. Early detection of preliminary stages of fever is not available, nor is temperature related seizure detection and responsive stimulation anticipated by any prior art.

Caloric stimulation (heating or cooling of the vestibular or balance mechanisms of the inner ear) has been shown to induce sleep or cause arousal (Abrams, R. M., et al., “Vestibular caloric responses and behavioral state in the fetal sheep,” Int. J Pediatri. Otorhinolaryngol. 1998, 45(1):59-68), modify sleep states (Velluti, R. A., et al., “Reciprocal actions between sensory signals and sleep,” Brol Signals Recpt, 2000, 9(6):297-308 and Cordero, L. et al., “Effects of vestibular stimulation on sleep states in premature infants,” Am. J Perinatol., 1986, 3(4):319-324) and reduce epileptic seizure activity in cats with longstanding effects (Guha, D. and Maiti, A. K., “Influences of vestibulo-cerebellum on kindling in the cat,” Indian J Med. Res., August 1989, 90:275-284). No device for therapeutic caloric stimulation is currently known to exist.

### SUMMARY OF THE INVENTION

In order to provide good electrographic signal sensing prior art such as the previously-referenced patent by Fischell et al. required electrodes implanted under the scalp. The present invention envisions use of non-invasive electrodes placed within the ear canal that may be combined with other surface electrodes. By placing electrodes into the ear canal in one or both ears it becomes possible to achieve multiple forms of recording and stimulation since the ear provides an anatomical aperture in the skull, greatly improving the quality of the EEG signal as compared with scalp electrodes. Such ear canal electrodes may achieve signal quality close to that of fully implanted electrodes. An additional advantage of the ear canal location is the close proximity of the ear canal to the vestibular structures of the inner ear allowing stimulation of vestibular mechanisms using electrical or caloric stimuli.

The caloric stimuli can be either heating or cooling using caloric transducers such as bimetallic strips, heat exchangers, and Peltier devices. Modes of operation include heating or cooling in one or both ears and heating and cooling together using the strips in various combinations. We envision that the bimetallic strips could also act as the electrodes for EEG recording and electrical stimulation and, that an acoustic transducer such as a piezoelectric crystal can be incorporated in the electrode/strip structure so that auditory detection and stimuli can also be provided.

A further advantage of this location is that the device can record changes in temperature within the skull and can apply responsive electrical, caloric or acoustic signals. Another advantage is that this is the only practical non-invasive method of therapeutic alteration of vestibular mechanisms. The same device can be used to provide responsive neurostimulation for detected seizure activity. The response may be electrical, caloric or acoustic.

Acoustic stimuli can include recorded speech, masking sounds, tones, random noise or soothing sounds such as waves breaking on the shore. Ideally, for waking or arousal alerting signals, a speech stimulus of the patient's name may be optimal. Loud alerting signals can also be employed without disturbing others.

Electrical stimuli can act as a simple sensory feedback stimulus as described by Fischell et al., but close proximity of the electrodes to the inner ear will probably allow induction of vestibular stimuli particularly in response to long duration slow frequency potentials. The electrical stimulation may be responsive to the detection of a neural or sleep related event. If not responsive, the stimulation may be continuous or periodic. The program of stimulation may be set by the physician, but may allow patient alteration of the program and/or patient initiation of specific stimuli.

The ear canal based present invention may connect to an electronic control module and/or other sensor either by wires or by wireless means. The wireless connections may allow short range (on the body) or long range communication at some distance from the ear canal device. Use of modern wireless technologies having currently-available chip sets such as using transmission protocols such as IEEE 802.11b, 802.11a or the “Bluetooth” standard would facilitate this aspect of the invention.

The device can be linked with extensive recording capability to provide the function of 24-hour Holter ECG, temperature and/or EEG (or other electrographic) monitoring. This would allow recording of neural information at home so that appropriate interventions can be planned. Further more, the effects of those interventions can also be monitored. The neural information can include seizure activity, sleep patterns, ECG for heart abnormalities and fever onset and resolution in adults but particularly in infants and small children.

A key use of the present invention is in the diagnosis and treatment of sleep disorders. In U.S. Pat. No. 6,016,449, Fischell et al. describe an implantable device with the capability of detecting the onset or precursor to a neurological event. For people with narcolepsy, the neurological event is the onset of sleep. What is more, it is clear that there is a detectable EEG pattern when people are falling asleep and for various stages of sleep (drowsiness, stage 1, 2, 3, 4, and REM). The present invention expands upon the Fischell concepts to specifically detect the EEG patterns associated with falling asleep and responsively alert the patient or apply a responsive therapy of acoustic energy, electrical stimulation, caloric stimulation and/or drug infusion using an electrically controlled patch release of chemicals that can be absorbed through the skin.

Results of vagal stimulation indicate that vestibular stimulation may have therapeutic effects on depression. Ear canal caloric or electrical stimulation can be continuous, periodic or patient initiated and can provide vestibular stimulation without requiring a permanent subcutaneously implanted device. Vestibular stimulation should have beneficial effects on dizziness, vertigo, seasickness and travel sickness (jet lag). Persistent effects of vestibular stimulation should decrease sensitivity to vestibular disorders such as Menieres disease and positional vertigo. The vestibular stimulation in this application could be triggered by an accelerometer or by electronystagmography (detection of eye movements in response to change of position)

It is also envisioned that a magnetic stimulator coil may be incorporated within the ear canal device to provide stimulation of the vestibular nerve and potentially other subdural neural structures. New toroidal technology and the fact that the ear canal location places the coil through the cranium makes it possible to produce a much smaller magnetic coil than is used in conventional transcranial magnetic stimulation devices. A small and discrete magnetic field should be sufficient to stimulate the vestibular nerve. Such magnetic stimulation of subdural brain stem structures may have a therapeutic effect on sleep, epilepsy, headache, migraine and pain including pain following stroke.

Upton and Longmire in 1975 showed that sensory feedback could stop seizure activity when detected from scalp EEG electrodes. Upton, A.R. et al., “The effects of feedback on focal epileptic discharges in man. A preliminary report,” Can. J Neurol. Sci., August 1975; 2(3):153-67. Auditory stimuli were effective in some patients. Electrical stimulation of the ear lobe was also effective. Vestibular stimulation was not tried but may also work. The present invention should have improved detection as the EEG signals in the ear canal should be less subject to artifact than scalp electrodes. The present invention that would incorporate the latest techniques in seizure detection should be far more effective in detecting and stopping seizures than the 30% success rate that was reported by Upton and Longmire.

Stimulating the vestibular nucleus via the vestibular nerve should have effects on the vagus nerve that is the main output of the vestibular nucleus. Such vagal stimulation has been shown to have a positive effect on controlling epileptic seizures and depression as well as the regulation of cardiac function.

It is also envisioned that the present invention may include an accelerometer attached to the head to detect head movement for the prevention of benign positional vertigo.

The term sensor as applied to the present invention includes, but is not limited to, electrodes for sensing electrical activity, temperature sensors, and accelerometers.

Thus, an objective of this invention is to provide an ear canal electrode sensor for use in the diagnosis and treatment of neurological, vestibular, and other disorders.

Another objective of this invention is to have the ear canal electrodes capable of use in electrical stimulation for therapeutic treatment of neurological, vestibular, and other disorders.

Another object of this invention is to provide an ear canal caloric transducer that can provide temperature detection in connection with caloric stimulation.

Still another object of the invention is to provide an acoustic transducer integrated with an ear canal electrode sensor the acoustic transducer capable of providing sound output in response to the detection of a neural event. The sound output could include white noise, tones, recorded speech such as the patient's name, instruction and soothing or alerting sounds.

Still another object of the invention is to provide an acoustic transducer integrated with an ear canal electrode sensor the acoustic transducer capable of picking up verbal instructions spoken by the patient to control function of an electronic device.

Still another object of the invention is to have the ear canal device connect by wires to an electronic control module having circuitry to diagnose and respond to neural events.

Still another object of the invention is to have the ear canal device connect by wireless means to an electronic control module having circuitry to diagnose and respond to neural events.

Still another object of the invention is to have the control module capable of recording data over time from the ear canal device. The data recorded can include EEG, ECG, temperature and pulse rate.

Still another object of the invention is to stimulate the vestibular nerve in order to produce changes in mood.

Still another object of the present invention is to include an accelerometer attached to the head to detect head movement for the prevention of benign positional vertigo and to detect nodding of the head with drowsiness. Such a device could provide a warning sensory signal to alert the subject during the operation of machinery, motor vehicles or potentially hazardous equipment.

Still another object of the present invention is to stimulate the vagus nerve via the vestibular nucleus to produce vagal effects on cardiac function thereby reducing arrhythmia and rapid heart beats.

Yet another object of the present invention is to have an integrated magnetic stimulation coil built in to the ear canal device.

These and other objects and advantages of this invention will become obvious to a person of ordinary skill in this art upon reading the detailed description of this invention including the associated drawings as presented herein.

### BRIEF DESCRIPTION OF THE DRAWINGS

FIG. 1 is a block diagram of the present invention system.

FIG. 2 is a sketch of an integrated single ear canal sensor/stimulator unit.

FIG. 3 is a sketch of an alternate embodiment of the present invention having a secondary control module that can be placed behind the ear like a hearing aid.

FIG. 4 is a diagram of the present invention linked by wireless means to a fully implantable responsive neurostimulator.

FIG. 5 is a diagram of the present invention linked by wired or wireless means to an electronically controlled drug releasing skin patch.

FIG. 6 is a diagram of the present invention linked to an eye movement (nystagmus) detecting device in a pair of eyeglasses.

### DETAILED DESCRIPTION OF THE INVENTION

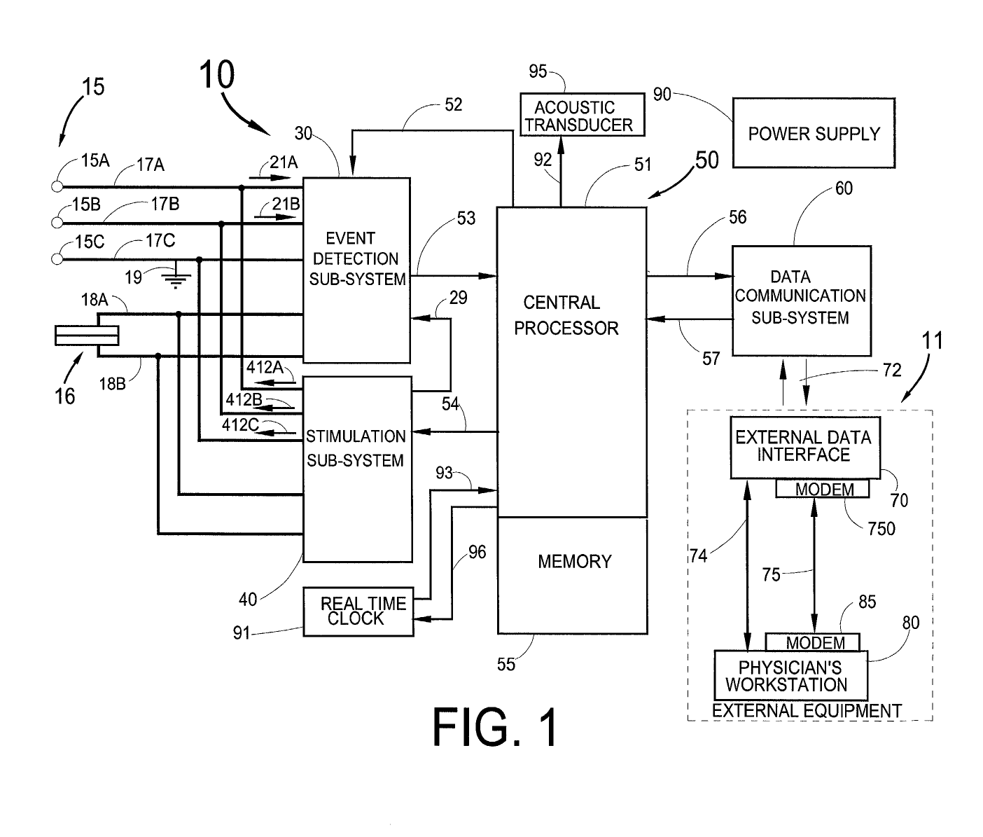

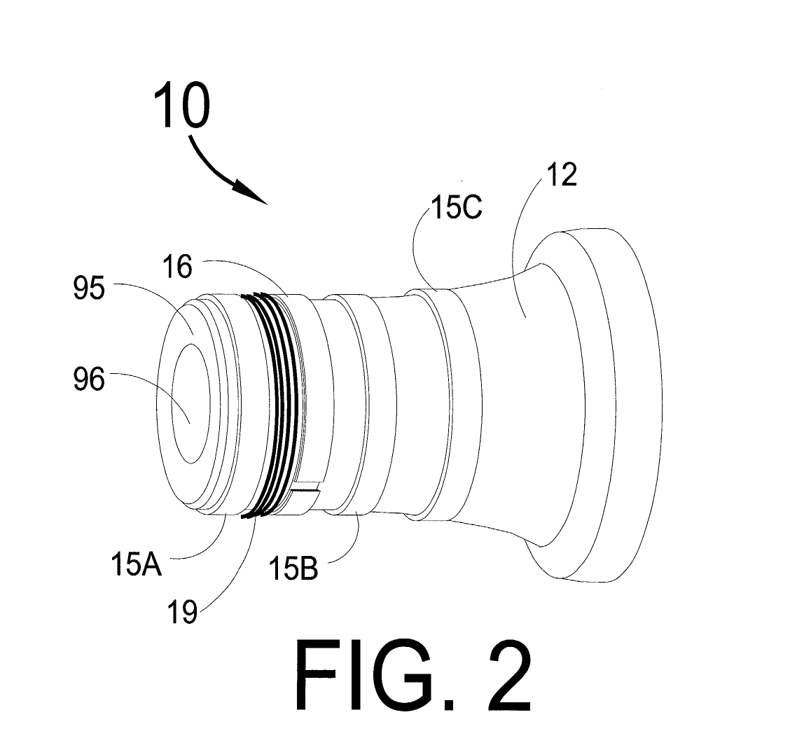

FIG. 1 is a block diagram of the ear canal sensor/stimulator system 10 and the external equipment 11. The wires 17A through 17C from the electrodes 15A through 15C, and the wires 18A and 18B from the caloric transducer 16, are shown connected to both the event detection sub-system 30 and the stimulation sub-system 40. 15C is designated as the common electrode although any of the electrodes could be so designated. It is also envisioned to use the case of the control module 20 of FIG. 2 as the common (or indifferent) electrode 15C. The wires 17A and 17B carry EEG signals 21A and 21B from the electrodes 15A and 15B to the event detection sub-system 30. The electrodes 15A and 15B can be energized by the stimulation sub-system 40 via the wires 17A through 17C to electrically stimulate the patient's vestibular nerve using the stimulation signals 412A through 412N respectively. Although the electrodes 15A through 15C shown here are connected to both the event detection sub-system 30 and the stimulation sub-system 40, it is obvious that a separate set of electrodes and associated wires could be used with each sub-system.

One envisioned embodiment of the caloric transducer is a bimetallic strip that can operate as a thermocouple to measure temperature or as a caloric stimulator to heat tissue in the vicinity of the patient's ear. It is contemplated that a bimetallic or resistive element can be used to heat the tissue, and a Peltier device or heat pump or exchanger can be used to either heat or cool the tissue. Other approaches are possible and will be recognized by a practitioner of ordinary skill in the art.

The event detection sub-system 30 receives the EEG signals 21A and 21B (referenced to system ground 19 connected to the wire 17C from the common electrode 15C and processes them to identify neurological events such as sleep state or an epileptic seizure or its precursor. A central processing system 50 with central processor 51 and memory 55 acts to control and coordinate all functions of the system 10. The interconnection 52 is used to transmit programming parameters and instructions to the event detection sub-system 30 from the central processing system 50. The interconnection 53 is used to transmit signals to the central processing system 50 identifying the detection of a neurological event by the event detection sub-system 30. The interconnection 53 is also used to transmit EEG and other related data for storage in the memory 55.

When an event is detected by the event detection sub-system 30, the central processor 51 can command the stimulation sub-system 40 via the interconnection 54 to transmit electrical signals to any one or more of the electrodes 15A and 15B via the wires 17A and 17B. It is anticipated that, if appropriate electrical signals 412A and 412B inclusive are transmitted to certain locations in or near the brain, the normal progression of an epileptic seizure can be aborted or sleep states may be modified. It may also be necessary for the stimulation sub-system 40 to temporarily disable the event detection sub-system 30 via the interconnection 29 when stimulation is imminent so that the stimulation signals are not inadvertently interpreted as a neurological event by the event detection system 30.

A power supply 90 provides power to each component of the system 10. Power supplies for comparable devices such as hearing aids are well known in the art of electronic devices. Such a power supply typically utilizes a primary (non-rechargeable) storage battery with an associated DC-to-DC converter to obtain whatever voltages are required for the system 10.

Data stored in the memory 55 can be retrieved by the patient's physician by a communication link 72 with the data communication sub-system 60 connected to the central processing system 50. The link 72 may be wireless or could use a physically wired connector. An external data interface 70 can be directly connected with an RS-232 type serial connection 74 to the physician's workstation 80. Alternately, the serial connection may be via modems 85 and 750 and phone line 75 from the patient's home to the physician's workstation 80. The software in the computer section of the physician's workstation 80 allows the physician to read out a history of events detected including EEG information before, during and after the event as well as specific information relating to the detection of the event such as the time evolving energy spectrum of the patient's EEG. The physician can also read out the level of arousal or the sleep stage. Such information could be used to detect the sleep stage associated with seizure activity, respiratory problems, cardiac problems, and other undesired symptoms and effects. The workstation 80 also allows the physician to specify or alter the programmable parameters of the system 10.

As shown in FIG. 1, an acoustic transducer 95 connected to the central processor 51 via the link 92 can be used to notify the patient that an event has occurred or that the system 10 is not functioning properly. This sound by itself can be a means for stopping an epileptic seizure as shown by Upton and Longmire in 1975, as described above. The acoustic transducer 95 can also act as a microphone to pick up spoken words used for patient device control via speech recognition software run by the central processor 51.

A real time clock 91 is used for timing and synchronizing various portions of the implanted system 10 and also to enable the system to provide the exact date and time corresponding to each neurological event that is detected by the system 10 and recorded in memory. The interconnection 96 is used to send data from the central processor 51 to the real time clock 91 in order to set the correct date and time in the clock 91.

The various interconnections between sub-systems (e.g., the interconnections 52, 53, 54, 56, 57, 92, 93 and 96) may be either analog or digital, single wire or multiple wires (a “data bus”).

The operation of the system 10 of FIG. 1 for detecting and treating a neurological event such as an epileptic seizure or inappropriate sleep state would be as follows:

1. The event detection sub-system 30 continuously processes the EEG signals 21A and 21B carried by the wires 17A and 17B from the electrodes 15A through 15N. The event detection sub-system may also monitor the patient temperature from the caloric transducer 16 placed in the ear canal.

2. When an event is detected, the event detection sub-system 30 notifies the central processor 51 via the link 53 that an event has occurred.

3. The central processor 51 then triggers the stimulation sub-system 40 via the link 54 to respond to the detected event with any combination of

a. electrical stimulation with either or both of the electrodes 15A and 15B,

b. caloric stimulation with the caloric transducer 16 to heat or cool the ear canal of the patient, and/or

c. acoustic stimulation from a sound played through the acoustic transducer 95.

4. The stimulation sub-system 40 also sends a signal via the link 29 to the event detection sub-system 30 to disable event detection during stimulation to avoid an undesired input into the event detection sub-system 30.

5. The central processor system 50 will store EEG signals, temperature historical data and event related data received from the event detection sub-system 30 via the link 53 over a time from X minutes before the event to Y minutes after the event for later analysis by the patient's physician. The value of X and Y may be set from as little as 0.1 minutes to as long as 30 minutes.

6. The central processor 51 may “buzz” to notify the patient that an event has occurred by sending a signal via the link 92 to the acoustic transducer 95. This “buzz” may be an auditory or tactile stimulation, and may in some situations be therapeutic in addition to informational.

Accordingly, it will be appreciated that one embodiment of the invention is adapted to provide responsive treatment for the disorders and symptoms described herein. However, the invention is not so limited, and may also provide continuous, semi-continuous, periodic, programmed, or on-demand (command-initiated) therapies, or various combinations of the foregoing, as clinically desired (and as described, for example, in U.S. patent application Ser. No. 09/543/450, filed on Apr. 5, 2000, which is hereby incorporated by reference as though set forth in full herein). The various treatment modalities set forth above (electrical stimulation, magnetic stimulation, acoustic and vibratory signals, heating and cooling, etc.) can also be applied individually or in combination as desired. See also U.S. Pat. No. 6,016,449 to Fischell et al. for further explanations of some of the above strategies.

Throughout FIGS. 1 through 6 inclusive, lines connecting boxes on block diagrams or on software flow charts will each be labeled with an element number. Lines without arrows between boxes and/or solid circles indicate a single wire.

Lines with arrows connecting boxes or circles are used to represent any of the following:

1. A physical connection, namely a wire or group of wires (data bus) over which analog or digital signals may be sent.

2. A data stream sent from one hardware element to another. Data streams include messages, analog or digital signals, commands, EEG information, and software downloads to change system operation and parameters.

3. A transfer of information between software modules. Such transfers include software subroutine calls with and without the passing of parameters, and the reading and writing of memory locations. In each case, the text will indicate the use of the line with an arrow.

FIG. 2 is a sketch of the mechanical structure of a single ear canal sensor/stimulator system 10 having an outer shell 12, electrodes 15A, 15B and 15C in the form of metallic rings, a caloric transducer 16 in the form of a bimetallic strip formed into a circular structure, magnetic stimulation coil 19, acoustic transducer 95 and acoustic channel 96. The acoustic channel 96 is a cylindrical hole that runs the length of the system 10 and allows sound from the outside to be transmitted through the device 10 to the eardrum of a human. The system 10 is adapted for placement into the ear canal and it is envisioned that the outer shape of the shell 12 could be formed (e.g., embedded in a custom-molded outer covering) to exactly fit a person's anatomy. The system 10 is completely self-contained with the electronics and battery enclosed within the shell 12. The apparatus would be constructed to allow cleaning and sterilization.

It should be noted that other types of sensors and transducers may be used in addition to or in place of the caloric transducer 16. For example, an accelerometer, a motion sensor, an orientation sensor, a blood pressure sensor, a blood flow sensor, a blood oxygenation sensor, a drug concentration sensor, a neurotransmitter concentration sensor, or a sleep sensor might be used in an embodiment of the invention to provide useful information for the detection of events or other purposes.

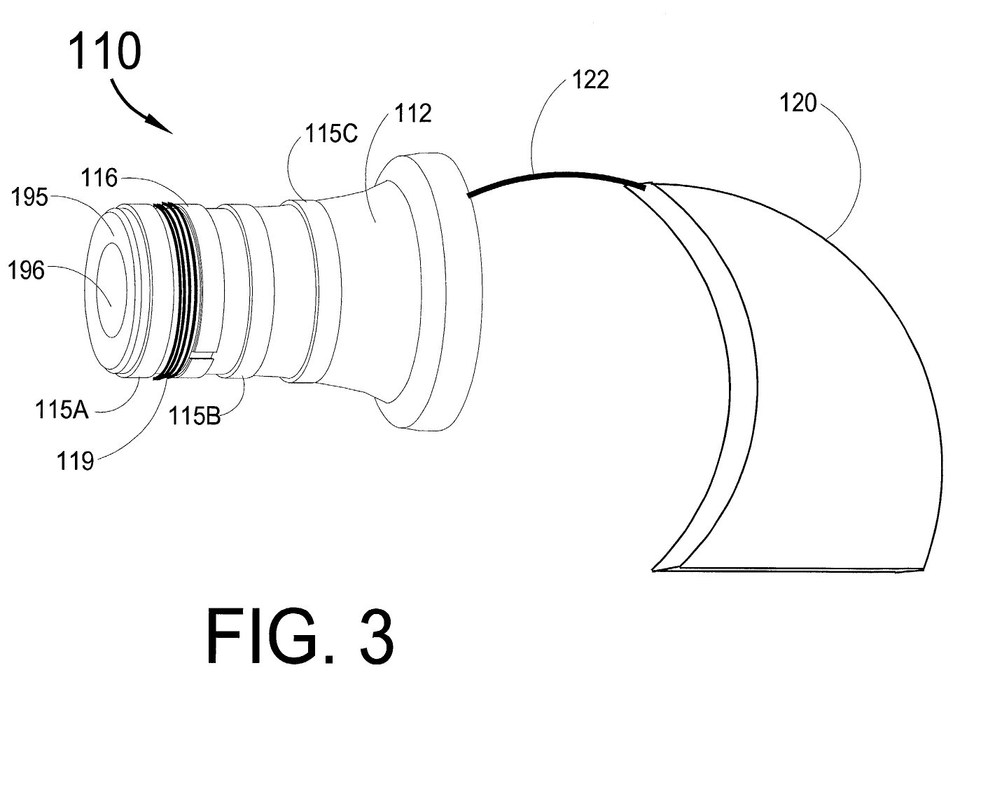

FIG. 3 is an alternate embodiment of the present invention 110 where similar to many hearing aids, the electronics are contained in a secondary control module 120 that would sit behind the ear and would be connected by the wire cable 122 to an ear sensor unit 110 having outer shell 112, electrodes 115A, 115B and 115C, caloric transducer 116, magnetic stimulation coil 119, acoustic transducer 195 and acoustic channel 196. These elements of the ear sensor unit 110 perform the same functions as those of the integrated ear canal system 10 of FIG. 2. It is also envisioned that wireless transmitters and receivers instead of the multi-wire cable 122 could connect the ear sensor unit 110 and control module 120. Such wireless transmission could use a standard protocol (such as Bluetooth or IEEE 802.11b), a custom protocol, or (for example) a wireless intrabody signaling technique to transmit data between the ear sensor unit 110 and the control module 120.

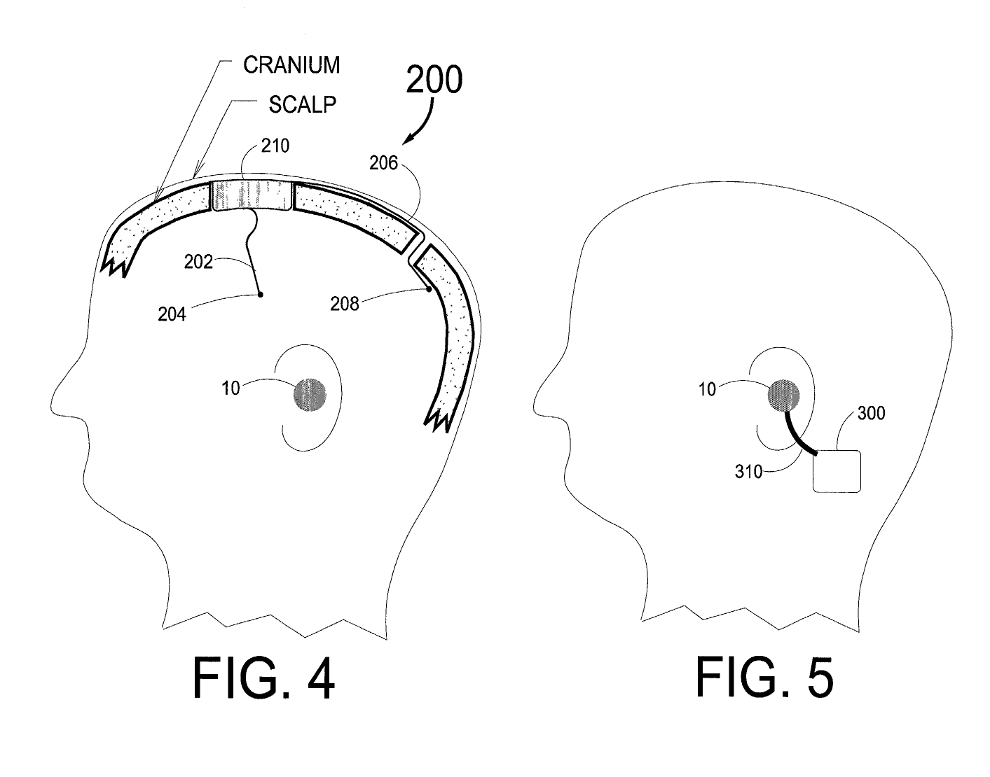

FIG. 4 shows still another application of the ear canal system 10 associated with a fully implanted responsive neurostimulator 210. The combined ear/implant system 200 would allow sensing or electrical stimulation in the brain using electrodes 204 and/or 208 connected by wires 202 and 206 to the implanted neurostimulator 210 in conjunction with the electrical, caloric, vestibular and acoustic sensing and stimulation capabilities of the ear canal system 10. This embodiment expands upon descriptions by Fischell et. al. in U.S. Pat. No. 6,016,449 where the ear device was only thought to provide acoustic stimulation. In certain neurological disorders it may become desirable to sense neurological events including inappropriate sleep states within the brain and use the ear canal system 10 to provide electrical, caloric and/or vestibular stimulation as a response to the detected event.

FIG. 5 is a diagram of the present invention ear canal sensor/stimulator 10 linked by the multi-wire cable 310 to an electronically controlled drug releasing skin patch 300. This embodiment would facilitate drug treatments responsive to events detected by the system 10. It is also envisioned that wireless transmitters and receivers instead of the cable 310 could connect the ear sensor unit 10 and implanted neurostimulator 210. Such wireless transmission could use a standard protocol such as Bluetooth or IEEE 802.11b or other techniques to transmit data between the ear sensor unit 10 and the neurostimulator 210. These data could include arousal state, seizure detection data, pre-seizure activity, and sleep state. Information from implanted devices could allow triggering of vestibular stimulation, auditory signals, and caloric stimulation. Therefore the ear device could act as a supplementary stimulator either alone or in combination with the internal or implanted stimulators of a fully implanted device for control of epilepsy, tremor, or sleep disorder.

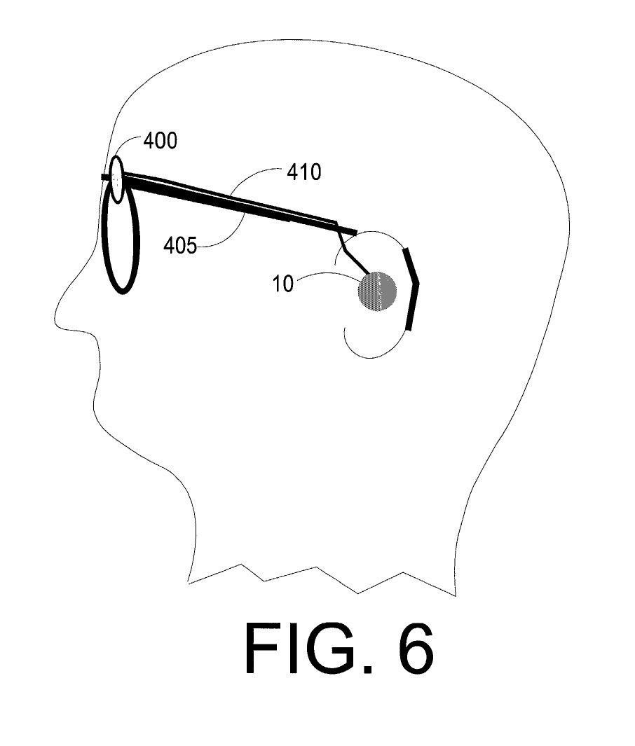

FIG. 6 is a diagram of the ear canal sensor/stimulator system 10 linked by the wire 410 to an eye movement (nystagmus) detecting device 400 in a pair of eyeglasses 405. The eyeglasses could include sensors that detect eye position such as electrode or miniature laser devices for detection of eye position, movement or nystagmus. Such information would help to detect dizziness, vertigo, rapid eye movement sleep and drowsiness. Detection of the position of the eye could involve bouncing a low energy laser beam off one or both eyes to a detector. It is also envisioned that additional surface electrodes in or on the arms could detect changes in the corneoretinal potentials as the eyes move. The glasses can also be used to provide a visual stimulus triggered by the ear device or an implanted stimulator. It is also envisioned that wireless transmitters and receivers instead of the cable 410 could connect the ear sensor unit 10 and eye movement device 400. Such wireless transmission could use a standard protocol such as Bluetooth or IEEE 802.11b or other techniques to transmit data between the ear sensor unit 10 and the device 400.

Various other modifications, adaptations and alternative designs are of course possible in light of the teachings as presented herein. Therefore it should be understood that, while still remaining within the scope and meaning of the appended claims, this invention could be practiced in a manner other than that which is specifically described herein.

What is claimed is:

1. A system for treating a neurological or vestibular disorder in a patient, the system comprising a stimulator device adapted to be situated in an ear canal of the patient for noninvasive interaction with the nervous system of the patient.

2. The system for treating a disorder of claim 1, wherein the noninvasive interaction comprises magnetic stimulation.

3. The system for treating a disorder of claim 2, wherein the magnetic stimulation is applied to a portion of the cerebral cortex of the patient.

4. The system for treating a disorder of claim 2, wherein the magnetic stimulation is applied to a vestibular nerve of the patient.

5. The system for treating a disorder of claim 1, wherein the noninvasive interaction comprises electrical stimulation applied with at least one electrode in contact with the ear canal.

6. The system for treating a disorder of claim 1, wherein the noninvasive interaction comprises an application of an audible or tactile signal.

7. The system for treating a disorder of claim 1, wherein the noninvasive interaction comprises caloric stimulation.

8. The system for treating a disorder of claim 1, wherein the stimulation device comprises a detection subsystem, and wherein the noninvasive interaction comprises performing an action in response to an event detected by the detection subsystem.

9. The system for treating a disorder of claim 8, wherein the detected event comprises a neurological event.

10. The system for treating a disorder of claim 8, wherein the detection subsystem is in communication with at least one sensor.

11. The system for treating a disorder of claim 10, wherein the sensor comprises an electrode, a temperature sensor, an accelerometer, a motion sensor, an orientation sensor, a blood pressure sensor, a blood flow sensor, a blood oxygenation sensor, a drug concentration sensor, a neurotransmitter concentration sensor, or a sleep sensor.

12. The system for treating a disorder of claim 8, wherein the detected event comprises an observation of a physiological condition indicated by a sensor measurement.

13. The system for treating a disorder of claim 8, wherein the event comprises an epileptic seizure, an onset of an epileptic seizure, or a precursor to an epileptic seizure.

14. The system for treating a disorder of claim 8, wherein the event comprises a sleep state transition.

15. The system for treating a disorder of claim 8, wherein the event comprises a symptom of positional vertigo.

16. The system for treating a disorder of claim 1, wherein the noninvasive interaction is performed on a continuous, semi-continuous, or programmed basis.

17. The system for treating a disorder of claim 1, wherein the noninvasive interaction is performed in response to a command.

18. The system for treating a disorder of claim 1, further comprising an external module in communication with the stimulator device.

19. The system for treating a disorder of claim 1, further comprising an implanted module in communication with the stimulator device.

20. The system for treating a disorder of claim 1, further comprising a second device adapted to be situated in a second ear canal of the patient.

21. The system for treating a disorder of claim 20, wherein the second device is in communication with the stimulator device.

22. A method for treating a neurological or vestibular disorder in a patient, with a system comprising a stimulator device situated in an ear canal of the patient, the method comprising the steps of:
detecting an event to be treated; and
applying a therapy with the stimulator device in response to the event.

23. The method for treating a disorder of claim 22, wherein the therapy is noninvasive.

24. The method for treating a disorder of claim 23, wherein the therapy comprises magnetic stimulation, electrical stimulation, caloric stimulation, tactile stimulation, or auditory stimulation.
